(19) United States
(12) Patent Application Publication
(10) Pub. No.: US 2025/0295906 A1
(43) Pub. Date: Sep. 25, 2025

(54) Body Fluid Flow Port

(71) Applicant: TERUMO KABUSHIKI KAISHA, Tokyo (JP)

(72) Inventors: Yu OSAWA, Kanagawa (JP); Kazuma YODA, Kanagawa (JP); Taishi NIIMI, Kanagawa (JP)

(73) Assignee: TERUMO KABUSHIKI KAISHA, Tokyo (JP)

(21) Appl. No.: 19/085,497

(22) Filed: Mar. 20, 2025

(30) Foreign Application Priority Data
Mar. 21, 2024 (JP) 2024-045205

(51) Int. Cl. A61M 39/02

(52) U.S. Cl. CPC A61M 39/0247; A61M 2039/025; A61M 2039/0261; A61M 2039/0276; A61M 2202/0208; A61M 2202/0464; A61M 2210/0693

(57) ABSTRACT

A body fluid flow port includes: a distal portion that is inserted into a containing cavity containing body fluid through a hole, includes a flow opening through which the body fluid flows, and includes a flow path that communicates with the flow opening and allows the body fluid to pass through the flow path; a fitting portion that is fitted into the hole, includes an internal space defined by a cylindrical outer wall and a cylindrical inner wall and connected to the flow path, and includes, on inside of the inner wall, an insertion portion that allows a medical device to pass into a cranium; and a proximal portion that is disposed outside a living body and includes a chamber connected to the internal space to receive the body fluid.

### CROSS-REFERENCE TO RELATED APPLICATION

This application is based on and claims priority to Japanese Patent Application No. 2024-045205 filed on Mar. 21, 2024, the entire content of which is incorporated herein by reference.

### TECHNOLOGICAL FIELD

The present invention generally relates to a body fluid flow port used for treatment of a brain disease.

### BACKGROUND DISCUSSION

Development of a brain disease such as cerebral infarction blocks blood flow for supplying oxygen to brain cells, which may cause a risk of damaging the brain cells. This is why cerebral infarction requires early reperfusion of blood flow. One treatment for the cerebral infarction described in WO 2023/181979 A proposes that fluid such as oxygenated cerebrospinal fluid is circulated by injecting the fluid into a body cavity in which cerebrospinal fluid of a patient is present and discharging the fluid to the outside of the body cavity, and oxygen is directly supplied to oxygen-deficient brain cells. The required amount of fluid such as oxygenated cerebrospinal fluid is larger than the amount of drug required for general treatment or treatment by administration of drug solution in anesthesia or the like. Therefore, it is necessary to provide an injection route and a discharge route in order to suppress pressure fluctuation in a cranium.

Typical locations for accessing a containing cavity containing cerebrospinal fluid from the outside of a living body include a lumbar spine, cerebral ventricles, and cisterns. However, adopting the lumbar spine, the cerebral ventricles, and the cisterns as the locations for accessing the containing cavity containing cerebrospinal fluid from the outside of the living body has the following advantages and disadvantages.

That is, in a case where the lumbar spine is adopted as the location for accessing the containing cavity for cerebrospinal fluid from the outside of the living body, there is an advantage that the containing cavity for cerebrospinal fluid can be accessed only by puncture with a device, imposing a small burden on a patient. On the other hand, since there is a certain distance from the lumbar spine as a puncture site to the brain as a treatment site, there is a disadvantage that it takes a certain amount of time to obtain the effect of fluid injection. Since a large fluid administration amount and a large flow rate are required for the treatment as described above, it is necessary to insert a medical device such as a catheter having an aperture with a size of 14 G (gauge) or more, but the lumbar spine has a narrow path. Thus, there is a disadvantage that puncture with a needle of 14 G or more is difficult. In addition, the treatment requires an injection route and a discharge route, but as described above, the lumbar spine has a narrow path. Thus, it is difficult to obtain both the injection route and the discharge route having a sufficient aperture.

In a case where the cerebral ventricles and the cisterns are adopted as the locations for accessing the containing cavity for cerebrospinal fluid from the outside of the living body, there is an advantage that the effect of fluid injection and discharge can be obtained early because the fluid is directly supplied to the brain. On the other hand, there is a disadvantage that it is necessary to puncture the head with a device or perform a craniotomy to remove a part of a cranial bone in order to access the cerebral ventricles and the cisterns, imposing a large burden on a patient. For example, it is necessary to insert two devices because the injection route and the discharge route are required. There is also a disadvantage that, from the cerebral ventricles and the cisterns, or in general access to a spinal cavity such as lumbar puncture, a discharge catheter that discharges fluid out of a body cavity may be occluded by a thrombus due to bleeding, tissue fragments, tissue sticking, or the like at the time of trepanation or puncture.

Disclosed here is a body fluid flow port capable of efficiently injecting and discharging fluid while reducing a burden on a patient.

### SUMMARY

The body fluid flow port is configured to be fitted into a hole formed on a surface of a living body, the body fluid flow port including: a distal portion that is insertable through the hole into a cavity in the living body containing body fluid, wherein the distal portion includes a flow opening through which the body fluid flows, and a flow path that communicates with the flow opening and allows the body fluid to pass through the flow path. The body fluid flow port also includes a fitting portion positionable in the hole and including an internal space defined by a cylindrical outer wall and a cylindrical inner wall, with the internal space being connected to the flow path. The fitting portion also includes, on inside of the inner wall, an insertion portion that allows a medical device to pass into a cranium. The body fluid flow port additionally includes a proximal portion that is disposed outside a living body and includes a chamber connected to the internal space to receive the body fluid.

According to the body fluid flow port described above (1), the distal portion passes through the hole formed on the living body surface to be inserted into the containing cavity containing the body fluid. The fitting portion is fitted into the hole formed on the living body surface, and includes, on the inside of the inner wall, the insertion portion that allows the medical device to pass into the cranium. The fitting portion also includes the internal space defined by the cylindrical outer wall and the cylindrical inner wall. The internal space of the fitting portion is connected to the flow path that allows fluid such as the body fluid flowing through the flow opening of the distal portion or a therapeutic agent to pass therethrough. The proximal portion is disposed outside the living body and includes the chamber. The chamber of the proximal portion is connected to the internal space of the fitting portion to receive the body fluid.

Therefore, according to the body fluid flow port described above, as the medical device for injecting fluid into the containing cavity for the body fluid, for example, an injection device is inserted through the insertion portion of the fitting portion to pass into the cranium, and the body fluid contained in the containing cavity is guided to and received in the chamber of the proximal portion from the flow opening of the distal portion through the flow path and the internal space of the fitting portion, so that the fluid can be injected and discharged by one hole formed on the living body surface. As a result, the body fluid flow port can reduce a burden on a patient.

In addition, a discharge route of the body fluid uses, not a catheter, but the flow path of the distal portion inserted into the containing cavity, the internal space of the fitting portion fitted into the hole formed on the living body surface, and the chamber of the proximal portion disposed outside the living body. This makes it possible to avoid occlusion of the discharge route of the body fluid. As a result, the body fluid flow port can efficiently inject and discharge the fluid. Note that the body fluid can be discharged by a catheter or the like, and the above-described discharge route of the body fluid can be used for injecting fluid such as drug solution into the containing cavity from the outside of the living body. Also in this case, the body fluid flow port has effects similar to the effects described above.

In the body fluid flow port, it may be preferable that the proximal portion further includes a communication opening that is connected to the chamber to communicate with outside of the chamber.

According to the body fluid flow port described above, the body fluid received in the chamber of the proximal portion reliably communicates with the outside of the chamber through the communication opening of the proximal portion. As a result, the body fluid flow port can more efficiently inject and discharge the fluid.

The flow path may preferably a first flow path, and the internal space is a second flow path that communicates with the outside of the living body.

The flow path of the distal portion that allows the body fluid flowing through the flow opening to pass therethrough is the first flow path. The internal space of the fitting portion defined by the cylindrical outer wall and the cylindrical inner wall is the second flow path that communicates with the outside of the living body. Therefore, the body fluid flowing through the flow opening smoothly flows through the first flow path and the second flow path, and is guided to the chamber of the proximal portion. As a result, the body fluid flow port can more efficiently inject and discharge the fluid.

The proximal portion may preferably be formed of a flexible material, and is deformable according to fluctuation in an internal pressure of the living body caused by the body fluid.

The proximal portion is deformable according to the fluctuation in the internal pressure of the living body caused by the body fluid. That is, the chamber of the proximal portion functions as a buffer that absorbs the fluctuation in the internal pressure of the living body, and can ensure compliance of the containing cavity containing the body fluid. The “compliance” refers to adaptability capable of maintaining the internal pressure of the living body by deformation or the like based on dynamic softness of the containing cavity (for example, a subarachnoid space) with respect to a change in internal volume of the living body to some extent.

The distal portion may preferably include a flared portion having a flared shape widening radially outward from the fitting portion, and an end of the flared portion has the flow opening.

Because the distal portion includes the flared portion having a flared shape widening radially outward from the fitting portion, the distal portion functions as an anchor after being inserted into the containing cavity for the body fluid. Therefore, when the body fluid flows from the flow opening at the end of the flared portion toward the flow path, the distal portion can stop the body fluid flow port from coming out of the hole formed on the living body surface. In addition, since the flow opening of the distal portion is provided at the flared distal end widening radially outward, the body fluid can be discharged from an entire circumference. This makes it possible to avoid occlusion of a flow route of the body fluid or the fluid to be injected to become unable to allow the fluid to flow by a thrombus due to bleeding, tissue fragments, tissue sticking, or the like at the time of forming the hole on the living body surface. As a result, the body fluid flow port of the above (5) can efficiently inject and discharge the fluid.

The distal portion may preferably include a plurality of branch portions having a shape branching and widening radially outward from the fitting portion, and an end of each of the branch portions has the flow opening.

Because the distal portion includes the plurality of branch portions having a shape branching and widening radially outward from the fitting portion, the distal portion functions as an anchor after being inserted into the containing cavity for the body fluid. Therefore, when the body fluid flows into the flow path from the flow openings at the ends of the branch portions or when the fluid such as drug solution is injected into the containing cavity from the outside of the living body through the chamber, the distal portion can stop the body fluid flow port from coming out of the hole formed on the living body surface. In addition, since the plurality of branch portions have a shape branching and widening radially outward from the fitting portion, a medical worker can easily insert the distal portion into the containing cavity for the body fluid through a hole opened by trepanation.

The fitting portion may preferably include a valve body that is provided in the insertion portion to block leakage of fluid inside the living body to the outside of the living body.

The valve body provided in the insertion portion for allowing the medical device to pass into the cranium blocks the leakage of the fluid inside the living body to the outside of the living body. Therefore, when the body fluid flow port is fitted into the hole formed on the living body surface, it is possible to suppress fluctuation in the internal pressure of the living body caused by the leakage of the fluid. Moreover, the valve body can reduce a risk of infection development without creating a subcutaneous tunnel by not allowing the body fluid present inside the containing cavity to come into contact with air. Furthermore, the valve body holds a device inserted through the insertion portion, so that the device can be fixed at a predetermined insertion position.

The hole may be a hole formed by trepanation of a head, and the body fluid is cerebrospinal fluid.

According to another aspect, a body fluid flow port is configured to be fitted into a hole in a head of a living body, with such hole extending from a surface of the head of the living body to a fluid-containing cavity in the living body. The body fluid flow port comprises: an axially extending fitting portion having an outer dimension configured to be positioned in the hole in the head of the living body when the body fluid flow port is fitted into the hole in the head; a distal portion that is at one axial end of the fitting portion and that is configured to be positioned in the fluid-containing cavity when the body fluid flow port is fitted into the hole in the head of the living body; and a proximal portion that is at an opposite axial end of the fitting portion and that is configured to be positioned exterior of the living body when the body fluid flow port is fitted into the hole in the head of the living body. The distal portion includes a flow opening that is in fluid communication with a flow path, with the flow opening allowing fluid in the fluid-containing cavity to flow into the flow path when the body fluid flow port is fitted into the hole in the living body. The proximal portion encloses an annular-shaped chamber, and the fitting portion includes an axially extending internal space that is in fluid communication with both the chamber in the proximal portion and the flow path in the distal portion so that fluid flowing into the flow opening when the body fluid flow port is fitted into the hole in the head of the living body flows into the chamber by way of the flow path and the internal space. The proximal portion is configured to permit the fluid received from the fluid-containing cavity by way of the flow opening, the flow path and the internal space to be discharged outside the chamber. The axially extending internal space surrounds an axially extending insertion path that is configured to receive a medical device so that the medical device is able to pass into the fluid-containing cavity in the head of the living body when the body fluid flow port is fitted into the hole in the head of the living body, the axially extending insertion path being closed at a portion along an axial extent of the insertion path to block leakage of the fluid in the fluid-containing cavity from flowing to outside the living body by way of the axially extending insertion path.

The body fluid flow port disclosed here is capable of efficiently injecting and discharging the fluid while reducing a burden on a patient.

Another aspect involves a method comprising positioning a body fluid flow port in a hole in a head of a living body, with the hole communicating with a fluid-containing cavity in the living body. The body fluid flow port includes: a distal portion, a proximal portion and a fitting portion extending between the distal portion and the proximal portion, with the fitting portion including a cylindrical outer wall and a cylindrical inner wall that are spaced apart from one another so that an internal space is located between the cylindrical outer wall and the cylindrical inner wall. The distal portion includes a flow opening and a flow path that is in fluid communication with both the flow opening and the internal space so that fluid in the fluid-containing cavity is able to enter the flow opening, flow along the flow path and flow into the internal space, the proximal portion including a chamber that is in fluid communication with the internal space so that the fluid in the internal space is able to flow into the chamber. The positioning of the body fluid flow port in the hole in the head of the living body comprises positioning the body fluid flow port so that: i) the fitting portion is positioned in the hole; ii) the proximal portion is positioned exterior of the living body so that at least a part of the chamber is exterior of the living body; and iii) the distal portion is positioned in the fluid-containing cavity so that the flow opening is in fluid communication with the fluid-containing cavity whereby the fluid in the fluid-containing cavity is able to enter the flow opening.

### BRIEF DESCRIPTION OF THE DRAWINGS

FIG. 1 is a schematic diagram illustrating a fluid circulation system in which a body fluid flow port according to the disclosure here is used.

FIG. 2 is a schematic diagram illustrating a fluid discharge system in which the body fluid flow port according to the present disclosure is used.

FIG. 3 is a perspective view illustrating a body fluid flow port according to a first embodiment.

FIG. 4 is a cross-sectional view taken along a cutting plane 4-4 illustrated in FIG. 3.

FIG. 5 is a cross-sectional view illustrating a body fluid flow port according to a first modification.

FIG. 6 is a cross-sectional view illustrating a body fluid flow port according to a second modification.

FIG. 7 is a perspective view illustrating a body fluid flow port according to a second embodiment.

FIG. 8 is a cross-sectional view taken along a cutting plane V8-8 illustrated in FIG. 7.

### DETAILED DESCRIPTION

Hereinafter, preferred embodiments of a fluid circulation/discharge system with a body fluid flow port will be described in detail with reference to the drawings representing examples of the new fluid circulation/discharge system and body fluid flow port disclosed here.

Embodiments described below are preferred examples, and so various technically preferable aspects are described. However, the scope of the invention is not limited to these aspects unless the following description states differently. Furthermore, in the drawings, similar components are denoted by the same reference numerals and so a detailed description of features and aspects already described are not repeated.

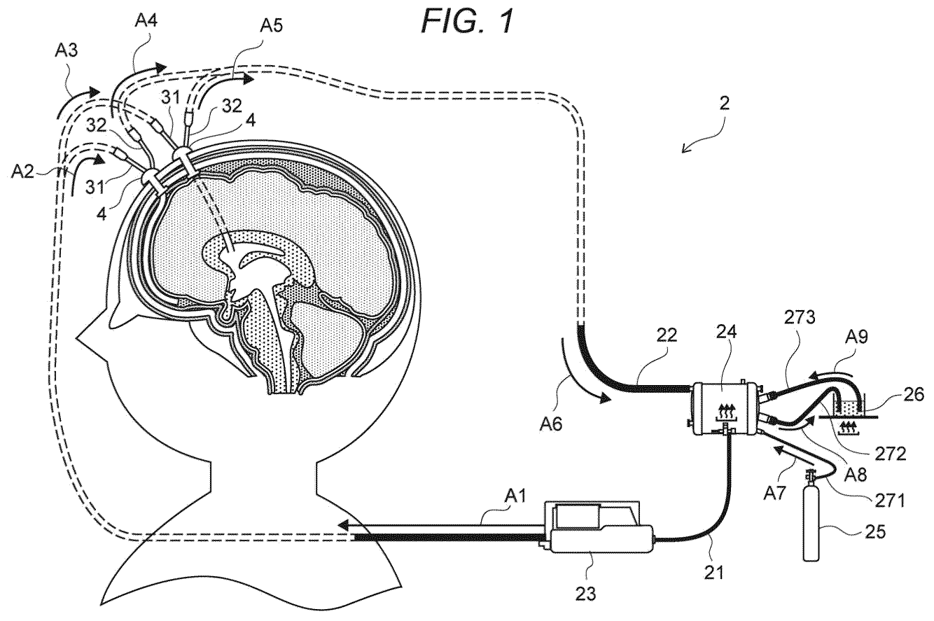

FIG. 1 is a schematic diagram illustrating a fluid circulation system in which a body fluid flow port according to the present embodiment is used.

A fluid circulation system 2 illustrated in FIG. 1 circulates fluid by injecting the fluid into a containing cavity containing cerebrospinal fluid (CSF) of a subject and discharging the fluid to the outside of the containing cavity. The cerebrospinal fluid is contained mainly in a subarachnoid space and cerebral ventricles. That is, the containing cavity containing the cerebrospinal fluid (fluid-containing cavity) includes the subarachnoid space and the cerebral ventricles. A part of the subarachnoid space includes cisterns such as a basal cistern, but a specific position is not particularly limited. The cerebrospinal fluid of the present embodiment is an example of “body fluid” in the present disclosure.

Examples of the fluid to be injected into the containing cavity include fluid having a higher oxygen concentration than an oxygen concentration of normal cerebrospinal fluid (that is, high oxygen solution). However, the fluid to be injected into the containing cavity is not limited to the high oxygen solution. For example, the fluid to be injected into the containing cavity may be fluid containing a drug and obtained by adding the drug to the cerebrospinal fluid during extracorporeal circulation, or may be cerebrospinal fluid filtered with a filter to remove an undesirable substance during extracorporeal circulation. In addition, the fluid to be injected into the containing cavity may be fluid obtained by performing certain processing, such as irradiation with energy or heating, on the cerebrospinal fluid.

Moreover, the fluid to be injected into the containing cavity in an initial stage of treatment may be artificial cerebrospinal fluid, or physiological saline or Ringer's lactate solution as a substitute for the cerebrospinal fluid. In the present embodiment, artificial cerebrospinal fluid, a substitute such as physiological saline or Ringer's lactate solution, a liquid mixture of artificial cerebrospinal fluid and a substitute such as physiological saline or Ringer's lactate solution, other drug solutions, and distilled water for injection may be collectively referred to as fluid. In the following description, an example in which the fluid to be injected into the containing cavity is the high oxygen solution may be given for convenience of description.

As illustrated in FIG. 1, the fluid circulation system 2 includes an injection line 21, a discharge line 22, a fluid feeder 23, and a body fluid flow port 4. The fluid circulation system 2 may include an oxygenation mechanism 24, an oxygen supply source 25, and a heat exchanger 26.

The injection line 21 includes an injection device 31 such as a catheter. The injection line 21 is inserted into the containing cavity through the body fluid flow port 4 located on the head of the subject to inject the fluid into the containing cavity as indicated by an arrow A1 illustrated in FIG. 1. As illustrated in FIG. 1, the injection line 21 is delivered to, for example, the cisterns such as a basal cistern, in the head, but is not particularly limited in this way as long as it is in the vicinity of a treatment target site such as a cerebral infarction lesion. In this case, the injection line 21 is inserted into the containing cavity through the body fluid flow port 4 as indicated by an arrow A2 illustrated in FIG. 1. A distal portion of the catheter or the like is delivered to a target site of the cistern along a brain surface, and the fluid is injected into the cistern by the indwelling injection device 31. Alternatively, as illustrated in FIG. 1, the injection line 21 may be delivered to, for example, the cerebral ventricles in the head. In this case, the injection line 21 is inserted into the containing cavity through the body fluid flow port 4 as indicated by an arrow A3 illustrated in FIG. 1. A distal portion of the catheter or the like is delivered to, for example, a lateral ventricle, and the fluid is injected into the ventricle by the indwelling injection device 31. The delivery of the injection device 31 may or may not penetrate brain parenchyma depending on a delivery position, and a preferred method is appropriately selected. For example, an access route used in common procedures of ventricular drainage and cisternal drainage is preferable.

The discharge line 22 includes a discharge device 32. The discharge line 22 is inserted into the body fluid flow port 4 or attached to a surface of the body fluid flow port 4. The containing cavity (for example, the subarachnoid space and the cerebral ventricles) containing the cerebrospinal fluid is a substantially closed space. A certain pressure such as intracranial pressure is applied to the inside of the containing cavity. Therefore, when the body fluid flow port 4 is located on the head and the discharge line 22 including the discharge device 32 is located in the body fluid flow port 4, the discharge line 22 discharges the fluid (for example, the cerebrospinal fluid) inside the containing cavity to the outside of the containing cavity as indicated by arrows A4 and A6 illustrated in FIG. 1 or arrows A5 and A6 illustrated in FIG. 1. At this time, a filter for filtering impurities, a reservoir for temporarily storing the cerebrospinal fluid and adjusting the intracranial pressure, and the like may be provided in the middle of the discharge line 22. The intracranial pressure of the present embodiment is an example of an “internal pressure of a living body” according to the present disclosure.

Details of the body fluid flow port according to the present embodiment will be described later.

The oxygenation mechanism 24 is connected to the oxygen supply source 25 through a first tube 271. The oxygenation mechanism 24 mixes oxygen supplied from the oxygen supply source 25 through the first tube 271 as indicated by an arrow A7 illustrated in FIG. 1 into the fluid such as the cerebrospinal fluid supplied through the discharge line 22 to generate oxygenated cerebrospinal fluid.

The oxygenation mechanism 24 is also connected to the heat exchanger 26 through a second tube 272 and a third tube 273. As indicated by an arrow A8 illustrated in FIG. 1, the oxygenation mechanism 24 supplies the oxygenated cerebrospinal fluid to the heat exchanger 26 through the second tube 272. The heat exchanger 26 adjusts a temperature of the cerebrospinal fluid supplied from the oxygenation mechanism 24 through the second tube 272. As indicated by an arrow A9 illustrated in FIG. 1, the heat exchanger 26 supplies the temperature-adjusted cerebrospinal fluid to the oxygenation mechanism 24 through the third tube 273. Then, the oxygenation mechanism 24 supplies the oxygenated temperature-adjusted cerebrospinal fluid as the high oxygen solution to the injection line 21. Examples of the oxygenation mechanism 24 include a hollow fiber membrane oxygenator for adding oxygen to blood.

The fluid feeder 23 is provided in the injection line 21 to circulate the fluid supplied from the oxygenation mechanism 24. The fluid feeder 23 may be provided in the discharge line 22 or may be provided in both the injection line 21 and the discharge line 22. Examples of the fluid feeder 23 include an infusion pump, a syringe pump, and a centrifugal pump. The fluid feeding method may be a method using free fall without using an infusion pump or the like.

As indicated by the arrow A1 illustrated in FIG. 1, the fluid feeder 23 feeds the fluid to the injection line 21 to inject the fluid from the head of the subject into the containing cavity through the injection line 21. As described above, the containing cavity (for example, the subarachnoid space and the cerebral ventricles) containing the cerebrospinal fluid is a substantially closed space. A certain pressure such as intracranial pressure is applied to the inside of the containing cavity. Therefore, when the injection line 21 injects the fluid into the containing cavity, the fluid (for example, the cerebrospinal fluid) inside the containing cavity is pushed out of the containing cavity through the discharge line 22 including the discharge device 32 as indicated by the arrows A4 and A6 illustrated in FIG. 1 or the arrows A5 and A6 illustrated in FIG. 1. In this manner, the fluid feeder 23 circulates the fluid.

Next, a fluid discharge system in which the body fluid flow port according to the present embodiment is used will be described.

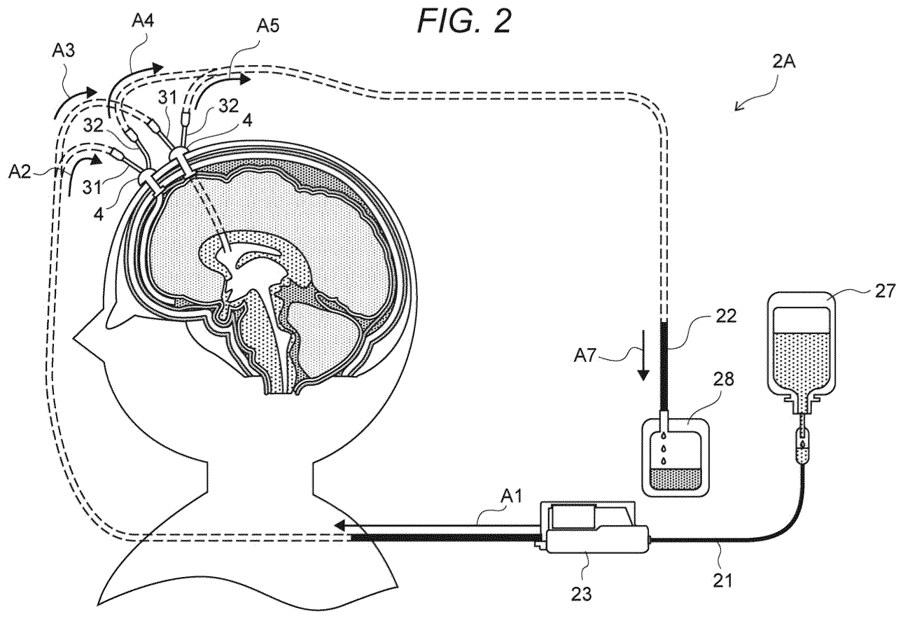

In a case where components of a fluid discharge system 2A described with reference to FIG. 2 are similar to the components of the fluid circulation system 2 described above with reference to FIG. 1, redundant description will be appropriately omitted, and differences will be mainly described hereinafter.

FIG. 2 is a schematic diagram illustrating the fluid discharge system in which the body fluid flow port according to the present embodiment is used.

The fluid discharge system 2A illustrated in FIG. 2 replaces the cerebrospinal fluid with the fluid by injecting the fluid into the containing cavity containing the cerebrospinal fluid of the subject and discharging the cerebrospinal fluid to the outside of the containing cavity.

As illustrated in FIG. 2, the fluid discharge system 2A includes the injection line 21, the discharge line 22, and the body fluid flow port 4. The fluid discharge system 2A may include the fluid feeder 23, a reservoir tank 27, and a storage unit 28.

As described above with reference to FIG. 1, the containing cavity (for example, the subarachnoid space and the cerebral ventricles) containing the cerebrospinal fluid is a substantially closed space. A certain pressure such as intracranial pressure is applied to the inside of the containing cavity. Therefore, when the body fluid flow port 4 is located on the head and the discharge line 22 including the discharge device 32 is located in the body fluid flow port 4, the discharge line 22 discharges the fluid (for example, the cerebrospinal fluid) inside the containing cavity to the outside of the containing cavity as indicated by arrows A4 and A7 illustrated in FIG. 2 or arrows A5 and A7 illustrated in FIG. 2. The cerebrospinal fluid discharged through the discharge line 22 is supplied to the storage unit 28 provided outside the living body.

The reservoir tank 27 stores the fluid to be injected into the containing cavity. Examples of the fluid stored in the reservoir tank 27 include highly-oxygenated artificial cerebrospinal fluid, as previously described with reference to FIG. 1 as the fluid to be injected into the containing cavity.

The fluid feeder 23 is provided in the injection line 21 to feed the fluid supplied from the reservoir tank 27. The fluid feeder 23 may be provided in the discharge line 22 or may be provided in both the injection line 21 and the discharge line 22. Examples of the fluid feeder 23 include an infusion pump, a syringe pump, and a centrifugal pump. The fluid feeder 23 is not necessarily provided, and the fluid may be fed by free fall or the like.

Other configurations and operations are similar to those of the fluid circulation system 2 described above with reference to FIG. 1.

Next, the body fluid flow port according to the present embodiment will be described in detail with reference to the drawings.

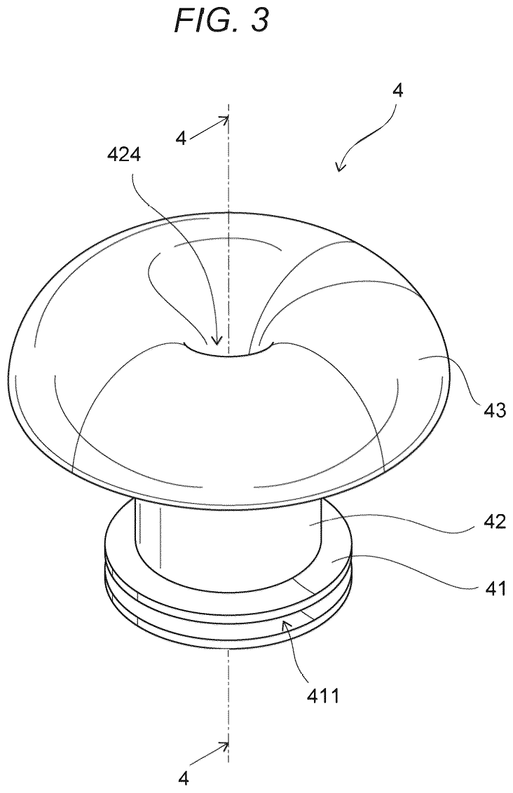

FIG. 3 is a perspective view illustrating a body fluid flow port according to a first embodiment.

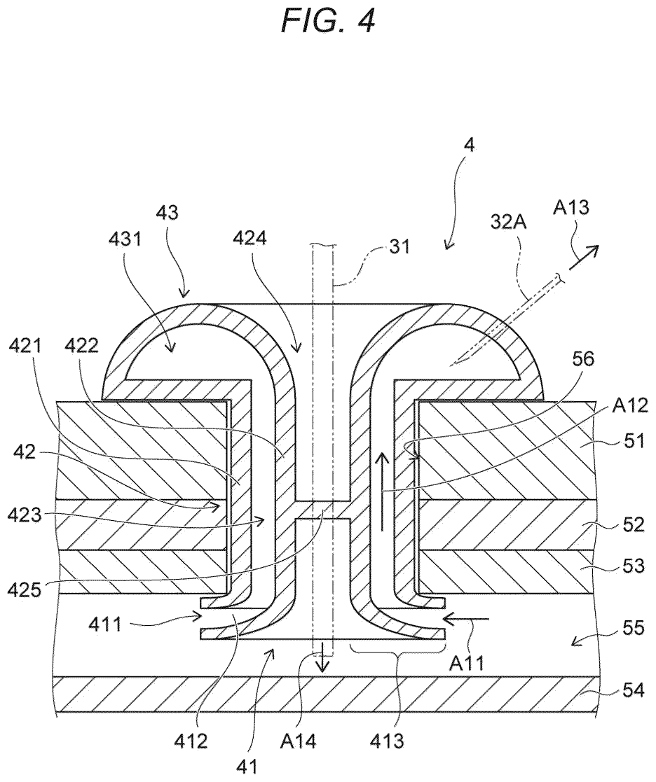

FIG. 4 is a cross-sectional view taken along a cutting plane (section line) 4-4 illustrated in FIG. 3.

The body fluid flow port 4 according to the present embodiment is formed of a flexible material, and is used by being fitted into a hole opened by trepanation. The “hole opened by trepanation” of the present embodiment is an example of a “hole formed on a living body surface” according to the disclosure here. The flexible material is not particularly limited as long as it is polyolefins such as polyethylene, polypropylene, polybutene, ethylene-propylene copolymer, ethylene-vinyl acetate copolymer, styrene-ethylene-butylene-styrene copolymer, ionomer, and a mixture containing at least two of these polyolefins, and thermoplastic resins such as soft polyvinyl chloride resin, polyamide, polyamide elastomer, nylon elastomer, polyester, polyester elastomer, polyurethane, and fluororesin, and also including silicone rubber and latex rubber. The flexible material is preferably silicone rubber.

For example, as illustrated in FIG. 4, the body fluid flow port 4 is inserted and fitted into a hole 56 opened by trepanation, that is, formed through a cranial bone 51, a dura mater 52, and an arachnoid mater 53. As illustrated in FIGS. 3 and 4, the body fluid flow port 4 includes a distal portion 41, a fitting portion 42, and a proximal portion 43. The fitting portion 42 is disposed between the distal portion 41 and the proximal portion 43 to be connected to the distal portion 41 on one side and connected to the proximal portion 43 on the other side. FIGS. 3 and 4 show that the outer dimension of both the distal portion 41 and the proximal portion 43 may be larger than the outer dimension of the fitting portion 42 so that the distal portion 41 and the proximal portion 43 extend further radially outwardly than the fitting portion 42.

The distal portion 41 passes through the hole 56 to be inserted into the containing cavity containing the cerebrospinal fluid. In the example illustrated in FIG. 4, the distal portion 41 passes through the hole 56 to be inserted into a subarachnoid space 55 between the arachnoid mater 53 and a pia mater 54. The distal portion 41 has a flared shape widening radially outward from the fitting portion 42, and includes a flow opening 411 and a flow path 412. In other words, the distal portion 41 includes a flared portion 413 having a flared shape widening radially outward from the fitting portion 42. At this time, since it is necessary to configure the distal portion 41 to be insertable into the containing cavity containing the cerebrospinal fluid, the flared shape preferably has a smaller thickness than a gap of the containing cavity. Because the flared shape has a smaller thickness than the gap of the containing cavity, the flow of the fluid in the distal portion 41 is not completely blocked, so that the injection and discharge of the fluid can be efficiently performed.

An end of the flared portion 413 has the flow opening 411. That is, the flow opening 411 is formed at the end of the flared portion 413. FIGS. 3 and 4 show that the flow opening 411 is located at a radially outwardly facing part of the flared portion so that the flow opening 411 faces radially outwardly. As indicated by an arrow A11 illustrated in FIG. 4, the cerebrospinal fluid present in the subarachnoid space 55 flows through the flow opening 411 formed in the distal portion 41 to flow into the inside of the distal portion 41. The flow path 412 communicates with the flow opening 411 and allows the cerebrospinal fluid flowing through the flow opening 411 to pass therethrough. The cerebrospinal fluid passing through the flow path 412 is guided to the fitting portion 42. The flow path 412 of the present embodiment is an example of a “first flow path” according to the present disclosure.

The fitting portion 42 is fitted into the hole 56 and includes a cylindrical outer wall 421 and a cylindrical inner wall 422. That is, as illustrated in FIG. 4, the fitting portion 42 has a double tube shape. FIG. 4 shows that the cylindrical outer wall 421 and the cylindrical inner wall 422 may be coaxial. An internal space 423 is formed between the cylindrical outer wall 421 and the cylindrical inner wall 422. The internal space 423 is a space defined by the outer wall 421 and the inner wall 422 (i.e., a space defined between the inner surface of the cylindrical outer wall 421 and the outer surface of the cylindrical inner wall 422), and is a space connected to the flow path 412 of the distal portion 41. As indicated by an arrow A12 illustrated in FIG. 4, the cerebrospinal fluid guided from the flow path 412 of the distal portion 41 passes through the internal space 423 of the fitting portion 42 to be guided to the proximal portion 43.

An insertion portion 424 is formed inside the inner wall 422 of the fitting portion 42. The insertion portion 424 functions as a path for allowing a medical device to pass into the cranium. The “medical device” referred to herein is, for example, the injection device 31 described above with reference to FIGS. 1 and 2. Examples of the injection device 31 include a catheter and a tube. The medical device passing through the insertion portion 424 may also be, for example, the discharge device 32 described above with reference to FIGS. 1 and 2. In this case, the fluid flows through the flow opening 411 to be injected into the subarachnoid space 55, and the cerebrospinal fluid is discharged to the outside of the subarachnoid space 55 by the discharge device 32 passing through the insertion portion 424. In the description of the present embodiment, a case where the injection device 31 passes through the insertion portion 424 will be described as an example.

The fitting portion 42 further includes a valve body 425. The valve body 425 is provided in the insertion portion 424 and is formed of a flexible material. The valve body 425 blocks leakage of the fluid (for example, the cerebrospinal fluid) inside the living body to the outside of the living body. As illustrated in FIG. 4, the valve body 425 allows the injection device 31 to pass therethrough. Therefore, as indicated by an arrow A14 illustrated in FIG. 4, the injection device 31 inserted into the insertion portion 424 and passing through the valve body 425 can inject the fluid into the subarachnoid space 55. Examples of a configuration of the valve body 425 include structural bodies such as a hemostasis valve and a torque device, and membranes of thermoplastic resins such as polyvinylidene chloride, vinyl chloride resin, polymethylpentene, polyethylene, and fluororesin, silicone rubber, and latex rubber. The configuration of the valve body 425 is not particularly limited as long as the leakage of the cerebrospinal fluid can be blocked.

The proximal portion 43 is disposed outside the living body and includes a chamber 431. The chamber 431 is connected to the internal space 423 of the fitting portion 42 to receive the cerebrospinal fluid passing through the internal space 423. A volume of the cerebrospinal fluid receivable in the chamber 431 may be any volume as long as the cerebrospinal fluid can be supported by strength of a joint portion between the fitting portion 42 and the proximal portion 43, and is about 1 mL to 50 mL. The proximal portion 43 is formed of a flexible material, and is deformable according to fluctuation in the intracranial pressure. That is, the chamber 431 of the proximal portion 43 functions as a buffer that absorbs the fluctuation in the intracranial pressure, and can ensure compliance of the containing cavity containing the cerebrospinal fluid. In the specification of the present application, the “compliance” refers to adaptability capable of maintaining the intracranial pressure by deformation or the like based on dynamic softness of the containing cavity (for example, the subarachnoid space 55) with respect to a change in intracranial volume to some extent.

As illustrated in FIG. 4, the proximal portion 43 is punctured with a needle 32A as an example of the discharge device 32 described above with reference to FIGS. 1 and 2, for example. A distal end of the needle 32A is disposed in the chamber 431. As indicated by an arrow A13 illustrated in FIG. 4, the cerebrospinal fluid received in the chamber 431 passes through a lumen of the needle 32A to be discharged to the outside of the living body.

Next, the fluid flow will be described.

When the body fluid flow port 4 is located on the head and the proximal portion 43 is punctured with the needle 32A, the cerebrospinal fluid present in the subarachnoid space 55 flows through the flow opening 411 of the distal portion 41 to flow into the distal portion 41, and flows through the flow path 412 of the distal portion 41, as indicated by the arrows A11 and A12 illustrated in FIG. 4. Alternatively, when the body fluid flow port 4 is located on the head, the proximal portion 43 is punctured with the needle 32A, and the fluid is injected into the subarachnoid space 55 through the injection device 31 as indicated by the arrow A14 illustrated in FIG. 4, the cerebrospinal fluid present in the subarachnoid space 55 flows through the flow opening 411 of the distal portion 41 to flow into the distal portion 41, and flows through the flow path 412 of the distal portion 41, as indicated by the arrows A11 and A12 illustrated in FIG. 4.

The cerebrospinal fluid having passed through the flow path 412 flows through the internal space 423 of the fitting portion 42 to be guided to the chamber 431 of the proximal portion 43. As indicated by the arrow A13 illustrated in FIG. 4, the cerebrospinal fluid received in the chamber 431 passes through the lumen of the needle 32A to be discharged to the outside of the living body.

In this manner, the internal space 423 of the fitting portion 42 functions as a flow path for discharging the cerebrospinal fluid to the outside of the living body. The internal space 423 of the present embodiment is an example of a “second flow path” according to the disclosure.

As described above, according to the body fluid flow port 4 of the present embodiment, as the medical device for injecting the fluid into the containing cavity for the cerebrospinal fluid, for example, the injection device 31 is inserted through the insertion portion 424 of the fitting portion 42 to pass into the subarachnoid space 55, and the cerebrospinal fluid contained in the containing cavity is guided to and received in the chamber 431 of the proximal portion 43 from the flow opening 411 of the distal portion 41 through the flow path 412 and the internal space 423 of the fitting portion 42, so that the fluid can be injected and discharged by trepanation at one position. As a result, the body fluid flow port 4 according to the present embodiment can reduce a burden on a patient.

In addition, a discharge route of the cerebrospinal fluid uses, not a catheter, but the flow path 412 of the distal portion 41 inserted into or positioned in the containing cavity, the internal space 423 of the fitting portion 42 fitted into the hole 56, and the chamber 431 of the proximal portion 43 disposed outside the living body. Since the flow opening 411 of the distal portion 41 is provided at the flared distal end widening radially outward, the cerebrospinal fluid can be discharged from an entire circumference. This makes it possible to avoid occlusion of the discharge route of the cerebrospinal fluid to become unable to discharge the cerebrospinal fluid by a thrombus due to bleeding, tissue fragments, tissue sticking, or the like at the time of trepanation. As a result, the body fluid flow port 4 according to the present embodiment can efficiently inject and discharge the fluid. In addition, any one of the injection and discharge can be performed on a deep region of the brain using the medical device such as a catheter inserted through the insertion portion 424, and the other one of the injection and discharge can be performed through the distal portion 41 disposed near a surface of the brain. This enables the injection location and the discharge location to be sufficiently separated from each other, to obtain a high therapeutic effect without immediately discharging the injected therapeutic fluid.

Moreover, since the internal space 423 of the fitting portion 42 functions as the flow path for discharging the cerebrospinal fluid to the outside of the living body, the cerebrospinal fluid flowing through the flow opening 411 of the distal portion 41 smoothly flows through the flow path 412 of the distal portion 41 and the internal space 423 of the fitting portion 42, and is guided to the chamber 431 of the proximal portion 43. As a result, the body fluid flow port 4 according to the present embodiment can more efficiently inject and discharge the fluid.

In addition, since the distal portion 41 includes the flared portion 413 having a flared shape widening radially outward from the fitting portion 42, the distal portion 41 functions as an anchor after being inserted into the subarachnoid space 55. Therefore, when the cerebrospinal fluid flows from the flow opening 411 at the end of the flared portion 413 toward the flow path 412, the distal portion 41 can stop the body fluid flow port 4 from coming out of the hole 56.

Furthermore, since the valve body 425 blocks the leakage of the fluid (for example, the cerebrospinal fluid) inside the living body to the outside of the living body, it is possible to suppress fluctuation in the intracranial pressure caused by the leakage of the fluid when the body fluid flow port 4 is fitted into the hole 56. Moreover, the valve body 425 can reduce a risk of infection development without creating a subcutaneous tunnel by not allowing the cerebrospinal fluid present inside the subarachnoid space 55 to come into contact with air. Furthermore, the valve body 425 holds the injection device 31 inserted through the insertion portion 424, so that the injection device 31 can be fixed at a predetermined insertion position.

Next, body fluid flow ports according to modifications of the present embodiment will be described with reference to the drawings.

Components of the body fluid flow ports according to the modifications of the present embodiment that are similar to the components of the body fluid flow port 4 according to the embodiment described above with reference to FIGS. 3 to 4 are identified by common reference numerals and a detailed description of such features is not repeated. The description of the modifications mainly focuses on differences relative to the earlier described embodiment and modifications.

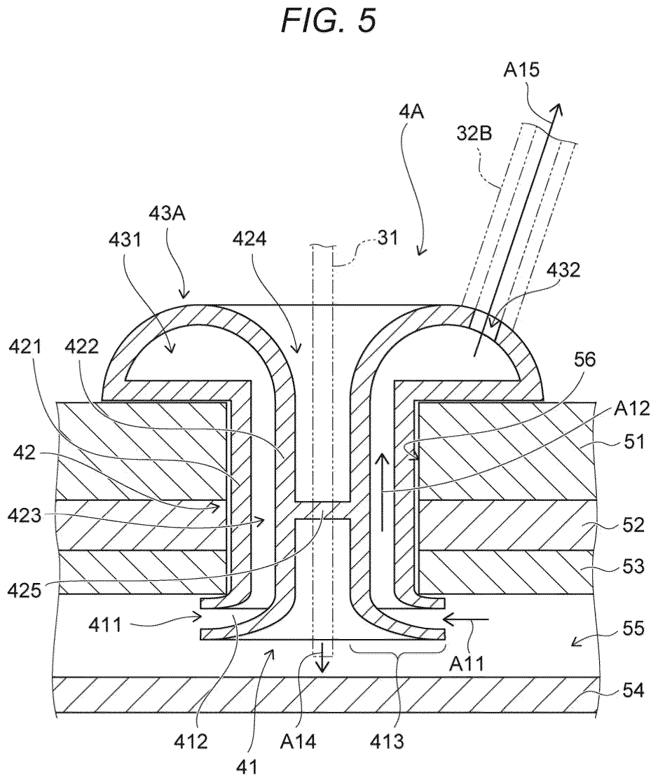

FIG. 5 is a cross-sectional view illustrating a body fluid flow port according to a first modification of the present embodiment.

FIG. 5 corresponds to the cross-sectional view taken along a cutting plane (section line) 4-4 similar to that illustrated in FIG. 3.

A body fluid flow port 4A according to the present specific example includes the distal portion 41, the fitting portion 42, and a proximal portion 43A. The distal portion 41 and the fitting portion 42 are as previously described with reference to FIGS. 3 and 4.

The proximal portion 43A includes a communication opening 432. The communication opening 432 connects the inside of the chamber 431 and the outside of the chamber 431 to discharge the cerebrospinal fluid received in the chamber 431 to the outside of the chamber 431. That is, the cerebrospinal fluid received in the chamber 431 passes through the communication opening 432 to be discharged to the outside of the chamber 431. As described above with reference to FIGS. 3 and 4, in a case where the fluid flows through the flow opening 411 to be injected into the subarachnoid space 55 (that is, in a case where the discharge device 32 passes through the insertion portion 424), the fluid passes through the communication opening 432 to flow into the chamber 431.

As illustrated in FIG. 5, for example, a tube 32B as an example of the discharge device 32 described above with reference to FIGS. 1 and 2 is attached to an outer surface of the proximal portion 43A. The tube 32B is not particularly limited as long as the tube 32B is attached so as to be able to discharge the cerebrospinal fluid without leaking the cerebrospinal fluid to the outside. Examples of the attachment method include a UV bonding method. A lumen of the tube 32B is connected to the communication opening 432. Therefore, as indicated by an arrow A15 illustrated in FIG. 5, the cerebrospinal fluid received in the chamber 431 passes through the communication opening 432 of the proximal portion 43A, flows through the lumen of the tube 32B, and is discharged to the outside of the living body.

Other features and structures of the body fluid flow port are similar to those of the body fluid flow port 4 described above with reference to FIGS. 3 to 4. The tube 32B may have a structure including a connector connectable to another medical device such as a catheter. Such a structure of the tube 32B makes it easy to perform an operation of executing a predetermined treatment on the cerebrospinal fluid after being discharged.

According to the body fluid flow port 4A of the present modification, since the communication opening 432 is provided in the proximal portion 43A to connect the inside of the chamber 431 and the outside of the chamber 431, the cerebrospinal fluid received in the chamber 431 of the proximal portion 43A is reliably discharged to the outside of the chamber 431 through the communication opening 432 of the proximal portion 43A. As a result, the body fluid flow port 4A according to the present specific example can more efficiently inject and discharge the fluid. In addition, effects similar to the effects of the body fluid flow port 4 described above with reference to FIGS. 3 to 4 can be obtained.

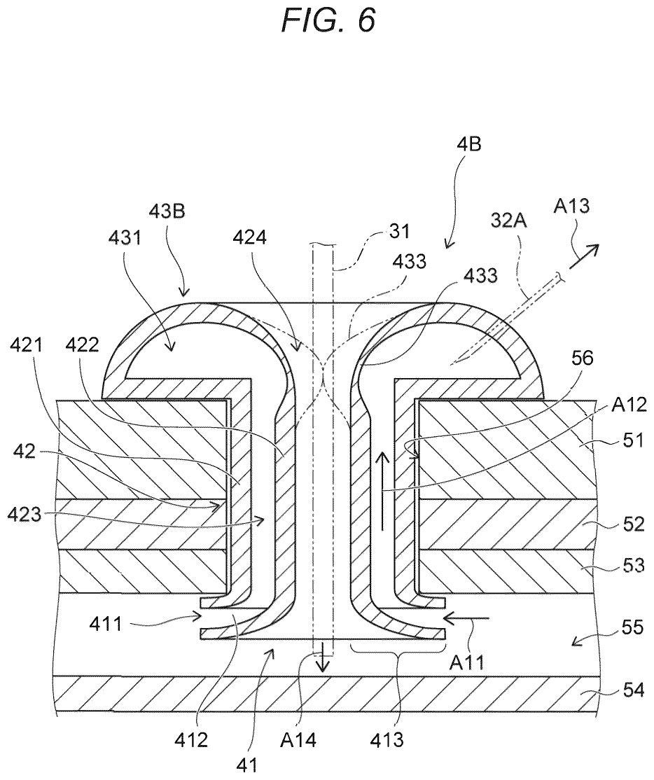

FIG. 6 is a cross-sectional view illustrating a body fluid flow port according to a second modification of the present embodiment.

FIG. 6 corresponds to the cross-sectional view taken along a cutting plane (section line) 4-4 similar to that illustrated in FIG. 3.

A body fluid flow port 4B according to the present specific example includes the distal portion 41, the fitting portion 42, and a proximal portion 43B. The distal portion 41 and the fitting portion 42 are as previously described with reference to FIGS. 3 and 4.

The proximal portion 43B includes an expansion portion 433. The expansion portion 433 is formed in an annular shape, for example, in the vicinity of an inlet of the insertion portion 424, and has a smaller thickness (thinned portion) than that of another portion of the proximal portion 43B. Therefore, when the cerebrospinal fluid is received in the chamber 431, the expansion portion 433 expands toward the inside of the inner wall 422, that is, the inside of the insertion portion 424 as indicated by a two-dot chain line illustrated in FIG. 6. Then, the insertion portion 424 is closed by the expansion portion 433. That is, the expansion portion 433 expands by the chamber 431 receiving the cerebrospinal fluid to close the insertion portion 424.

In this manner, the expansion portion 433 functions similarly to the valve body 425 described above with reference to FIG. 4 by expansion, and can block the leakage of the fluid (for example, the cerebrospinal fluid) inside the living body to the outside of the living body. As a result, according to the body fluid flow port 4B of the present specific example, the expansion portion 433 can reduce a risk of infection development without creating a subcutaneous tunnel by not allowing the cerebrospinal fluid present inside the subarachnoid space 55 to come into contact with air. In addition, effects similar to the effects of the body fluid flow port 4 described above with reference to FIGS. 3 to 4 can be obtained. Furthermore, in the body fluid flow port 4B according to the second modification, a communication opening and a tube as in the body fluid flow port 4A according to the first modification illustrated in FIG. 5 may be provided.

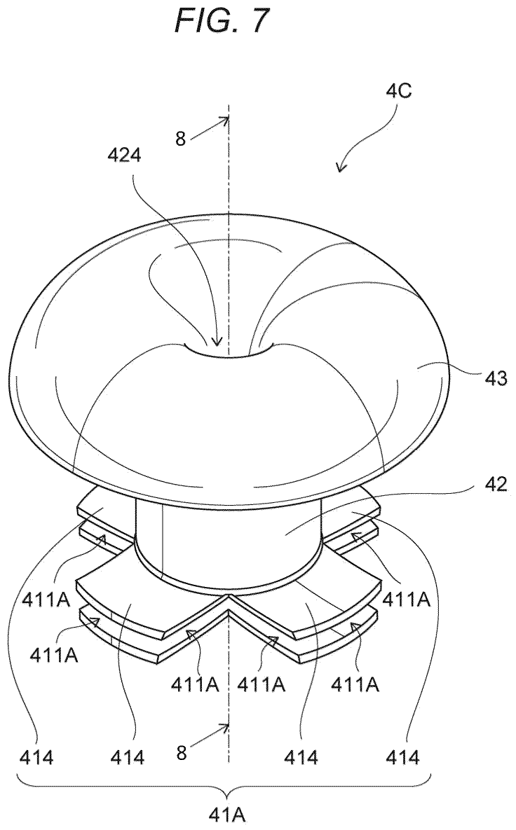

FIG. 7 is a perspective view illustrating a body fluid flow port according to a second embodiment.

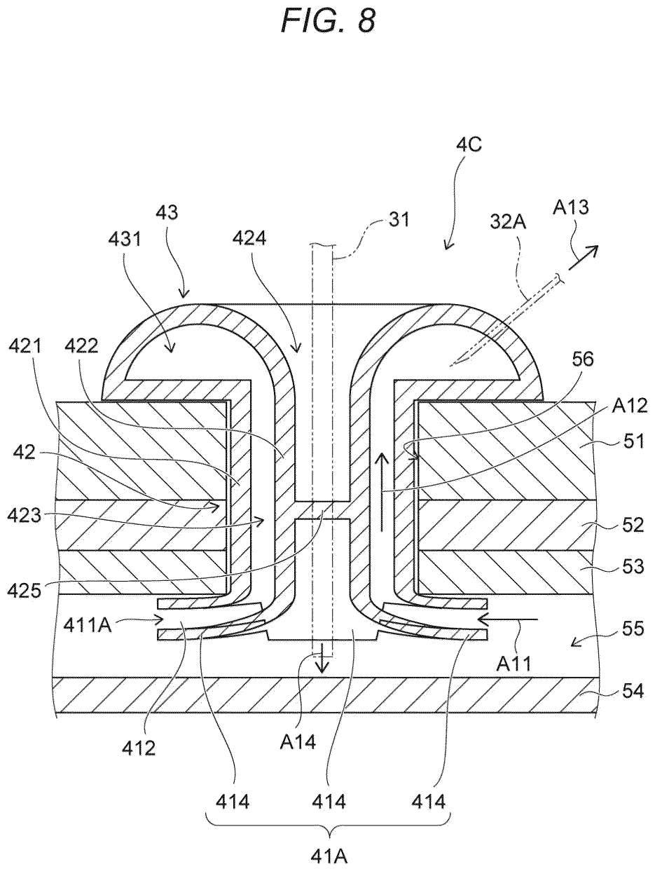

FIG. 8 is a cross-sectional view taken along a cutting plane (section line) 8-8 illustrated in FIG. 7.

Components or features of a body fluid flow port 4C according to the present embodiment that are similar to the components or features of the body fluid flow port 4 according to the present embodiment described above with reference to FIGS. 3 to 4 are identified by common reference numerals and a detailed description of such components and features is not repeated. The description of this modification mainly focuses on differences relative to the earlier described embodiment and modifications.

The body fluid flow port 4C includes a distal portion 41A, the fitting portion 42, and the proximal portion 43. The fitting portion 42 and the proximal portion 43 are as previously described with reference to FIGS. 3 and 4.

The distal portion 41A includes a plurality of branch portions 414. In the body fluid flow port 4C illustrated in FIG. 7, the distal portion 41A includes four branch portions 414. However, the number of branch portions 414 is not limited to 4, and may be 2, 3, or 5 or more.

The branch portions 414 have a shape branching and widening radially outward from the fitting portion 42. By configuring the branch portions 414 in this manner, it becomes easy to perform an operation such as folding the distal portion 41A, making it easy to insert the distal portion 41A into the containing cavity containing the cerebrospinal fluid. An end of each branch portion 414 has a flow opening 411A. As illustrated in FIG. 7, in the specification of the present application, the “end of each branch portion 414” includes not only a distal end of the branch portion 414 toward the radially outward from the fitting portion 42, but also ends on the opposite sides of the branch portion 414 toward the radially outward from the fitting portion 42. In the body fluid flow port 4C illustrated in FIG. 7, the flow opening 411A is formed at the distal end of the branch portion 414 toward the radially outward from the fitting portion 42 and at the ends on the opposite sides of the branch portion 414 toward the radially outward from the fitting portion 42.

Other structures are similar to those of the body fluid flow port 4 described above with reference to FIGS. 3 to 4.

According to the body fluid flow port 4C of the present embodiment, since the distal portion 41A includes the plurality of branch portions 414 having a shape branching and widening radially outward from the fitting portion 42, the distal portion 41A functions as an anchor after being inserted into the subarachnoid space 55. Therefore, when the cerebrospinal fluid flows into the flow path 412 from the flow openings 411A at the ends of the branch portions 414 or when the fluid such as drug solution is injected into the containing cavity from the outside of the living body through the chamber 431, the distal portion 41A can stop the body fluid flow port 4C from coming out of the hole 56. In addition, since the plurality of branch portions 414 have a shape branching and widening radially outward from the fitting portion 42, a medical worker can easily insert the distal portion 41A into the subarachnoid space 55 through the hole 56. In addition, effects similar to the effects of the body fluid flow port 4 described above with reference to FIGS. 3 to 4 can be obtained. The body fluid flow port 4C according to the second embodiment can have a configuration similar to those of the modifications illustrated in FIGS. 5 to 6. When the body fluid flow port 4C has the configuration similar to those of FIGS. 5 to 6, effects similar to the effects described above with reference to FIGS. 5 to 6 can be obtained.

Embodiments of the fluid circulation/discharge system and body fluid flow port have been described above. However, the present invention is not limited to the embodiments disclosed, and various modifications can be made without departing from the scope of the claims. The configurations of the embodiments may be partially omitted or optionally combined to make different configurations from the aforementioned configurations. In the embodiments, the flow path 412 serves as a discharge route, and the insertion portion 424 serves as an injection route, but these functions may be reversed. That is, the fluid may be injected into the living body from the flow path 412, and the body fluid may be discharged by inserting the discharge device 32 through the insertion portion 424.

The detailed description above describes embodiments of a fluid circulation/discharge system and body fluid flow port representing examples of the new fluid circulation/discharge system and body fluid flow port disclosed here. The invention is not limited, however, to the precise embodiments, modifications and variations described. Various changes, modifications and equivalents can be effected by one skilled in the art without departing from the spirit and scope of the invention as defined in the accompanying claims. It is expressly intended that all such changes, modifications and equivalents that fall within the scope of the claims are embraced by the claims.

What is claimed is:

1. A body fluid flow port configured to be fitted into a hole in a head of a living body, with such hole extending from a surface of the head of the living body to a fluid-containing cavity in the head, the body fluid flow port comprising:
an axially extending fitting portion having an outer dimension configured to be positioned in the hole in the head of the living body when the body fluid flow port is fitted into the hole in the head;
a distal portion that is at one axial end of the fitting portion and that is configured to be positioned in the fluid-containing cavity when the body fluid flow port is fitted into the hole in the head of the living body;
a proximal portion that is at an opposite axial end of the fitting portion and that is configured to be positioned exterior of the living body when the body fluid flow port is fitted into the hole in the head of the living body;
the distal portion including a flow opening that is in fluid communication with a flow path, the flow opening allowing fluid in the fluid-containing cavity to flow into the flow path when the body fluid flow port is fitted into the hole in the head of the living body;
the proximal portion enclosing an annular-shaped chamber;
the fitting portion including an axially extending internal space that is in fluid communication with both the chamber in the proximal portion and the flow path in the distal portion so that fluid flowing into the flow opening when the body fluid flow port is fitted into the hole in the head of the living body flows into the chamber by way of the flow path and the internal space;
the proximal portion being configured to permit the fluid received from the fluid-containing cavity by way of the flow opening, the flow path and the internal space to be discharged outside the chamber; and
the axially extending internal space surrounding an axially extending insertion path that is configured to receive a medical device so that the medical device is able to pass into the fluid-containing cavity in the head of the living body when the body fluid flow port is fitted into the hole in the head of the living body, the axially extending insertion path being closed at a portion along an axial extent of the insertion path to block leakage of the fluid in the fluid-containing cavity from flowing to outside the living body by way of the axially extending insertion path.

2. The body fluid flow port according to claim 1, wherein the fitting portion includes a cylindrical inner wall that has an outer surface and a cylindrical outer wall that is coaxial with the cylindrical inner wall and that has an inner surface, the outer surface of the inner wall and the inner surface of the outer wall being spaced apart from one another so that the axially extending internal space is located between the outer surface of the inner wall and the inner surface of the outer wall.

3. The body fluid flow port according to claim 2, wherein the cylindrical inner wall has an inner surface surrounding the insertion path.

4. The method according to claim 1, wherein the distal portion is a flared portion that flares radially outwardly.

5. The method according to claim 4, wherein the flow opening is located at a radially outwardly facing part of the flared portion so that the flow opening faces radially outwardly.

6. A body fluid flow port configured to be fitted into a hole formed on a surface of a living body, the body fluid flow port comprising:
a distal portion that is insertable through the hole and into a cavity in the living body containing body fluid, the distal portion including a flow opening through which the body fluid flows, the distal portion also including a flow path that communicates with the flow opening and allows the body fluid to pass through the flow path;
a fitting portion that is positionable in the hole, the fitting portion including an internal space defined by a cylindrical outer wall and a cylindrical inner wall, the internal space being connected to the flow path, the fitting portion including, on inside of the inner wall, an insertion portion that allows a medical device to pass into a cranium; and
a proximal portion that is positionable outside the living body and includes a chamber connected to the internal space to receive the body fluid.

7. The body fluid flow port according to claim 6, wherein the proximal portion further includes a communication opening that is connected to the chamber to communicate with outside of the chamber.

8. The body fluid flow port according to claim 6, wherein
the flow path is a first flow path, and
the internal space is a second flow path that communicates with the outside of the living body.

9. The body fluid flow port according to claim 6, wherein the proximal portion is formed of a flexible material, and is deformable according to fluctuation in an internal pressure of the living body caused by the body fluid.

10. The body fluid flow port according to claim 6, wherein
the distal portion includes a flared portion having a flared shape widening radially outward from the fitting portion, and
the flow opening is provided in an end of the flared portion.

11. The body fluid flow port according to claim 6, wherein
The flow opening is one of plural flow openings;
the distal portion includes a plurality of branch portions having a shape branching and widening radially outward from the fitting portion, and
an end of each of the branch portions has one of the flow openings.

12. The body fluid flow port according to claim 6, wherein the fitting portion includes a valve body that is provided in the insertion portion to block leakage of fluid inside the living body to the outside of the living body.

13. The body fluid flow port according to claim 6, wherein the chamber in the proximal portion is an annular chamber.

14. A method comprising:
positioning a body fluid flow port in a hole in a head of a living body, the hole communicating with a fluid-containing cavity in the living body, the body fluid flow port comprising:
a distal portion, a proximal portion and a fitting portion extending between the distal portion and the proximal portion, the fitting portion including a cylindrical outer wall and a cylindrical inner wall that are spaced apart from one another so that an internal space is located between the cylindrical outer wall and the cylindrical inner wall, the distal portion including a flow opening, the distal portion including a flow path that is in fluid communication with both the flow opening and the internal space so that fluid in the fluid-containing cavity is able to enter the flow opening, flow along the flow path and flow into the internal space, the proximal portion including a chamber that is in fluid communication with the internal space so that the fluid in the internal space is able to flow into the chamber; and
the positioning of the body fluid flow port in the hole in the head of the living body comprising positioning the body fluid flow port so that: i) the fitting portion is positioned in the hole; ii) the proximal portion is positioned exterior of the living body so that at least a part of the chamber is exterior of the living body; and iii) the distal portion is positioned in the fluid-containing cavity so that the flow opening is in fluid communication with the fluid-containing cavity whereby the fluid in the fluid-containing cavity is able to enter the flow opening.

15. The method according to claim 14, wherein the fluid-containing cavity is a subarachnoid space in the living body.

16. The method according to claim 14, wherein the cylindrical inner wall has an inner surface surrounding an insertion portion configured to allow a medical device to pass into a cranium of the living body by way of the insertion portion, the insertion portion extending completely through the fitting portion.

17. The method according to claim 16, further comprising a valve body extending across the insertion portion to block leakage of the fluid in the fluid-containing cavity to the exterior of the living body.

18. The method according to claim 14, wherein the distal portion is a flared portion that flares radially outwardly.

19. The method according to claim 18, wherein the flow opening is located at a radially outwardly facing part of the flared portion so that the flow opening faces radially outwardly.

20. The method according to claim 14, wherein the proximal portion includes a thinned part that is annular in shape and that has a thickness less than an immediately adjoining part of the proximal portion.
